(19) United States
(12) Patent Application Publication
(10) Pub. No.: US 2018/0368686 A1
(43) Pub. Date: Dec. 27, 2018

(54) APPARATUS AND METHOD FOR GENERATING A FUSED SCAN IMAGE OF A PATIENT

(71) Applicant: The Governors of the University of Alberta, Edmonton (CA)

(72) Inventors: Kumaradevan PUNITHAKUMAR, Edmonton (CA); Harald BECHER, Edmonton (CA); Pierre BOULANGER, Edmonton (CA); Michelle NOGA, Edmonton (CA); Abhilash Rakkunedeth HAREENDRANATHAN, Edmonton (CA)

(21) Appl. No.: 16/062,171

(22) PCT Filed: Dec. 14, 2016
(86) PCT No.: PCT/CA2016/051475

§ 371 Date: Jun. 14, 2018

Related U.S. Application Data
(60) Provisional application No. 62/267,054, filed on Dec. 14, 2015.

(51) Int. Cl. A61B 5/00; A61B 5/0452; A61B 5/113; A61B 8/08; A61B 8/14; A61B 8/00; A61B 90/00; A61B 34/20

(52) U.S. Cl. CPC A61B 5/0035; A61B 2090/378; A61B 5/0077; A61B 5/0452; A61B 5/113; A61B 5/726; A61B 5/7285; A61B 8/0883; A61B 8/145; A61B 8/4245; A61B 8/466; A61B 8/483; A61B 90/37; A61B 34/20; A61B 2034/2051; A61B 2034/2055; A61B 5/0064

(57) ABSTRACT

An apparatus and method for generating a fused scan image from a plurality of anatomical scan images of a patient. A tracking system is used to track the physical position and orientation of a scanner transducer, such as an ultrasound probe, which is used to obtain the anatomical scan images. The tracking system may also be used to track markers positioned on the patient for tracking anatomical movement of the patient. An image-processing based fusion is applied to the plurality of anatomical scan images based on the tracked position and orientation of the scanner transducer and the tracked patient anatomical movement to generate a fused scan image. The anatomical movement may comprise respiratory movement of the patient. An electrocardiogram (ECG) signal from the patient may be used to time synchronize tracking information generated by the tracking system with the plurality of anatomical scan images.

### CROSS REFERENCE TO RELATED APPLICATIONS

This application claims the benefit of priority of U.S. Provisional Patent Application No. 62/267,054 filed on Dec. 14, 2015, which is incorporated herein by reference.

### FIELD

The present disclosure relates to anatomical imaging. More specifically, the present disclosure relates to anatomical imaging wherein multiple image scans are fused to generate a fused scan image.

### BACKGROUND

Anatomical imaging is used to produce two dimensional and three dimensional images. One type of anatomical imaging is echocardiography. Echocardiography is used for image modality for cardiac functional analysis and image-guided interventions. The advantages of echocardiography include lack of ionizing radiation, portability, low cost, and higher temporal resolution compared to other modalities. Recent developments in ultrasound technology have enabled three-dimensional (3D) acquisitions of the heart, which allow visualization of the complex cardiac anatomy, and analysis of the complex combination of cardiac motions in 3D space.

Major limitations of 3D echocardiography in comparison to computed tomography (CT) and magnetic resonance imaging (MRI) include limited field of view (FOV), reliance on frequently limited acoustic windows, and poor signal to noise ratio (SNR). Due to limited FOV, a single 3D echocardiography acquisition may not be sufficient to cover the whole geometry of the heart. Previous methods attempted to solve the problem by acquiring multiple single-view images with small transducer movements and using image registration to align them. One disadvantage of using an image registration algorithm is that it requires sufficient overlap between images to produce accurate alignment. In general, image registration algorithms are computationally expensive. Additionally, the accuracy of alignment is bounded by the image resolution which is approximately one millimeter for a typical 3D ultrasound image. Further, ultrasound images are prone to speckle noise, and therefore, relying on image information may lead to inaccurate alignment.

Other approaches to fusion rely on image registration for initial calibration of the tracking system. Therefore, the aforementioned problems related to image registration may affect the accuracy of the image alignment.

The above information is presented as background information only to assist with an understanding of the present disclosure. No assertion or admission is made as to whether any of the above might be applicable as prior art with regard to the present disclosure.

### SUMMARY

According to an aspect, the present disclosure is directed to an apparatus comprising at least one scanner transducer in communication with an anatomical scanner and configured to generate a plurality of anatomical scan images of a patient, a tracking system comprising one or more sensors for tracking a position and orientation of the at least one scanner transducer, and patient anatomical movement, and a processor configured to receive signals from the tracking system and the plurality of anatomical scan images from the anatomical scanner, the processor further configured to apply image-processing based fusion to the plurality of anatomical scan images based on the tracked position and orientation of the at least one scanner transducer and the tracked patient anatomical movement to generate a fused scan image.

In an embodiment, the apparatus comprises an electrocardiogram (ECG) sensor configured to generate an ECG signal from the patient, and wherein the processor is further configured to time synchronize tracking information generated by the tracking system with the plurality of anatomical scan images based on the ECG signal.

In an embodiment, the anatomical movement is respiratory movement.

In an embodiment, the apparatus comprises an electrocardiogram (ECG) sensor configured to generate an ECG signal from the patient, wherein the processor is further configured to compute an overall average of the patient respiratory displacement based on the tracked patient respiratory movement during multiple previous R-R intervals in the ECG signal, select a subset of the plurality anatomical scan images, each anatomical scan image in the subset having been taken during an R-R interval that has an interval average patient respiratory displacement that is within a predefined threshold of the computed average patient respiratory displacement, generate the fused scan image from the selected subset of the plurality anatomical scan images.

In an embodiment, the processor is further configured to compute an interval variance of the patient respiratory displacement based on the tracked patient respiratory movement during each R-R interval in the ECG signal, the variance being a difference between the maximum and minimum tracked displacement values within the given R-R interval, and where each anatomical scan image in the subset having been taken during an R-R interval that has a computed variance patient respiratory displacement under a predefined variance value.

In an embodiment, the apparatus further comprises an electrocardiogram (ECG) sensor configured to generate an ECG signal from the patient, wherein the processor is further configured to select a subset of the plurality anatomical scan images, each anatomical scan image in the subset having been taken during a same subinterval of a respective R-R interval based on the ECG signal where the same subinterval corresponds to a particular phase of a heartbeat, generate the fused scan image from the selected subset of the plurality anatomical scan images.

In an embodiment, the image-processing based fusion is a wavelet based image fusion.

In an embodiment, the image-processing based fusion is a random walker image fusion.

In an embodiment, the scanner transducer is an ultrasound transducer.

In an embodiment, the tracking system comprises at least one mechanical tracking system comprising at least one measuring arm for tracking at least one of the position of the scanner transducer and the anatomical movement of the patient.

In an embodiment, the at least one measuring arm is configured for tracking the position and orientation of the scanner transducer, and the apparatus further comprises an optical tracking system comprising a plurality of cameras for tracking one or more patient markers positioned at the patient for tracking the patient anatomical movement.

In an embodiment, the tracking system comprises an optical tracking system comprising a plurality of cameras for tracking at least one of one or more scanner transducer markers positioned at the scanner transducer and one or more patient markers positioned at the patient for tracking the patient anatomical movement.

In an embodiment, the tracking system comprises an electromagnetic tracking system comprising a one or more electromagnetic sensors for tracking at least one of the position and orientation of the scanner transducer and the patient anatomical movement.

In an embodiment, alignment of the plurality of anatomical scan images during the generating of the fused scan image is performed independent of image data of the plurality of anatomical scan images.

In an embodiment, the apparatus is configured to generate the fused scan image in the form of a three dimensional echocardiography image.

According to an aspect, the present disclosure is directed to a method comprising generating a plurality of anatomical scan images of a patient with at least one scanner transducer, tracking a position and orientation of the at least one scanner transducer during the generating, tracking patient anatomical movement during the generating, and applying image-processing based fusion to the plurality of anatomical scan images based on the tracked position and orientation of the at least one scanner transducer and the tracked patient anatomical movement to generate a fused scan image.

In an embodiment, the method further comprises generating an electrocardiogram (ECG) signal from the patient, and time synchronizing tracking information generated by the tracking system with the plurality of anatomical scan images based on the ECG signal.

In an embodiment, the anatomical movement is respiratory movement.

In an embodiment, the method further comprises generating an electrocardiogram (ECG) signal from the patient, computing an overall average of the patient respiratory displacement based on the tracked patient respiratory movement during multiple previous R-R intervals in the ECG signal, selecting a subset of the plurality anatomical scan images, each anatomical scan image in the subset having been taken during an R-R interval that has an interval average patient respiratory displacement that is within a predefined threshold of the computed average patient respiratory displacement, generating the fused scan image from the selected subset of the plurality anatomical scan images.

In an embodiment, the method further comprises computing an interval variance of the patient respiratory displacement based on the tracked patient respiratory movement during each R-R interval in the ECG signal, the variance being a difference between the maximum and minimum tracked displacement values within the given R-R interval, and where each anatomical scan image in the subset having been taken during an R-R interval that has a computed variance patient respiratory displacement under a predefined variance value.

In an embodiment, the method further comprises generating an electrocardiogram (ECG) signal from the patient, selecting a subset of the plurality anatomical scan images, each anatomical scan image in the subset having been taken during a same subinterval of a respective R-R interval based on the ECG signal where the same subinterval corresponds to a particular phase of a heartbeat, generating the fused scan image from the selected subset of the plurality anatomical scan images.

In an embodiment, the image-processing based fusion is a wavelet based image fusion.

In an embodiment, the image-processing based fusion is a random walker image fusion.

In an embodiment, the plurality of anatomical scan images is generated with an ultrasound transducer.

In an embodiment, the tracking of at least one of the position of the scanner transducer and the anatomical movement of the patient is performed using a measuring arm.

In an embodiment, the tracking of the position and orientation of the scanner transducer is performed using the measuring arm, the method further comprising tracking the anatomical movement of the patient using an optical tracking system comprising a plurality of cameras for tracking one or more patient markers positioned at the patient.

In an embodiment, the tracking of at least one of the position and orientation of the scanner transducer and the anatomical movement of the patient is performed using an optical tracking system comprising a plurality of cameras for tracking at least one of one or more scanner transducer markers positioned at the scanner transducer and one or more patient markers positioned at the patient for tracking the patient anatomical movement.

In an embodiment, the tracking of at least one of the position and orientation of the scanner transducer and the anatomical movement of the patient is performed using an electromagnetic tracking system comprising one or more electromagnetic sensors for tracking at least one of the position and orientation of the scanner transducer and the patient anatomical movement.

In an embodiment, alignment of the plurality of anatomical scan images during the generating of the fused scan image is performed independent of image data of the plurality of anatomical scan images.

In an embodiment, the method generates the fused scan image in the form of a three dimensional echocardiography image.

According to an aspect, the present disclosure is directed to an apparatus comprising at least one scanner transducer in communication with an anatomical scanner and configured to generate a plurality of echocardiography scan images of a patient, a tracking system comprising one or more sensors for tracking a position and orientation of the at least one scanner transducer, and patient respiratory movement, an electrocardiogram (ECG) sensor configured to generate an ECG signal from the patient, and a processor configured to receive the plurality of echocardiography scan images from the anatomical scanner and signals from the tracking system, time synchronize tracking information generated by the tracking system with the plurality of echocardiography scan images based on the ECG signal, apply wavelet based image fusion to the synchronized plurality of echocardiography scan images based on the tracked position and orientation of the at least one scanner transducer and the tracked patient respiratory movement to generate a fused three dimensional echocardiography scan image.

According to an aspect, the present disclosure is directed to a method comprising generating a plurality of echocardiography scan images of a patient with at least one scanner transducer, tracking a position and orientation of the at least one scanner during the generating, tracking patient respiratory movement during the generating, generating an electrocardiogram (ECG) signal from the patient, time synchronizing tracking information with the plurality of echocardiography scan images based on the ECG signal, the tracking information being generated by the tracking the position and orientation of the at least one scanner transducer and the tracking the patient respiratory movement, and applying wavelet based image fusion to the synchronized plurality of echocardiography scan images based on the tracked position and orientation of the at least one scanner transducer and the tracked patient respiratory movement to generate a fused three dimensional echocardiography scan image.

The foregoing summary provides some aspects and features according to the present disclosure but is not intended to be limiting. Other aspects and features of the present disclosure will become apparent to those ordinarily skilled in the art upon review of the following description of specific embodiments in conjunction with the accompanying figures. Accordingly, the drawings and detailed description are to be regarded as illustrative in nature and not restrictive.

### BRIEF DESCRIPTION OF THE DRAWINGS

Embodiments of the present disclosure will now be described, by way of example only, with reference to the attached Figures.

FIGS. 1A and 1B are plan views illustrating the position of the heart changing between inspiration and expiration, respectively, relative to a fixed position of the probe with respect to the patient.

FIG. 2A is a side view a set of markers attached to an ultrasound transducer that tracked in 3D space by a multi-camera optical tracking system.

FIG. 2B is a plan view illustrating the movement of ultrasound probe during two different scans that can be combined for enhanced field of view.

FIG. 3 is a block diagram of an embodiment of a medical imaging system configured to perform the image fusion method of the present disclosure.

FIG. 4A illustrates a patient with a plurality of optical respiratory markers and electrocardiogram (ECG) electrodes secured to the patient's body.

FIG. 4B illustrates an approach to estimating positions of the respiratory markers over the respiratory cycle by computing the normal distances to a regression plane estimated using the initial positions of all respiratory markers.

FIG. 5 is a graph illustrating average displacement over all respiratory markers at each time step.

FIG. 6 is a perspective view of a wireframe model of an echocardiography transducer obtained using a laser scanner.

FIG. 7 is a diagram showing steps in the wavelet-based fusion algorithm of the present disclosure.

FIG. 8 is a side view of a system for estimating patient movement during image acquisition.

FIGS. 9A and 9B are echocardiography sequences with large spatial separation of 3D volumes before and after fusion, respectively.

FIG. 10 are graphs illustrating the marker displacement and ECG signals for fusion of data sets with free breathing and continuous acquisition.

FIGS. 11A-C illustrate image volumes taken from three orthogonal planes before applying the algorithm of the present disclosure.

FIGS. 12A-C illustrate image volumes after applying the wavelet fusion algorithm of the present disclosure.

FIGS. 13A and 13B illustrate example images showing manually demarcated septal and blood pool in long-axis and short-axis views, respectively.

FIGS. 14A-F illustrate single images (FIGS. 14A, 14B, 14D, 14E) and images fused (FIGS. 14C and 14F) according to the present disclosure.

FIG. 15 is a block diagram of an embodiment of a medical imaging system, comprising a mechanical tracking system, configured to perform the image fusion method of the present disclosure.

FIG. 16 is a close-up view of the mechanical tracking system of FIG. 15.

FIG. 17 is a diagram of an example measuring arm that may be used in the mechanical tracking system.

FIG. 18 is an example 3 dimensional image showing the fusion of multiple echocardiography scans where the scanner transducer placements were tracked using a measuring arm.

FIG. 19 is a block diagram of an embodiment of a medical imaging system, comprising a electromagnetic tracking system, configured to perform the image fusion method of the present disclosure.

FIG. 20 is a surface representation of a scanner transducer and electromagnetic sensors obtained using a laser scan.

FIG. 21 is an example 3 dimensional image showing the fusion of multiple echocardiography scans where the scanner transducer placements were tracked using an electromagnetic tracking system.

FIG. 22 is a graph of a representative example of the sum of absolute difference (SAD) versus an artificially introduced translation in x, y, z coordinate directions from the obtained alignment.

FIG. 23 is a process flow chart for generating a fused scan image according to an embodiment.

FIG. 24 is a block diagram of an example electronic device that may be used in implementing one or more aspects or components of an embodiment.

While the present disclosure is amenable to various modifications and alternative forms, specific embodiments have been shown by way of example in the drawings and are described in detail below. The intention, however, is not to limit the present disclosure to the particular embodiments described. On the contrary, the present disclosure is intended to cover all modifications, equivalents, and alternatives falling within the scope of the invention as defined by the appended claims.

### DETAILED DESCRIPTION

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

Some approaches to fusion use an optical tracking device or an electromagnetic tracking system to align the ultrasound images. However, many of these approaches rely on image registration for initial calibration of the tracking system. Therefore, problems related to image registration may affect the accuracy of the image alignment.

Imaging of anatomical structures using medical scanning devices often involves the sequential acquisition of data from different portions of the region being imaged. These acquisitions can sometimes be performed in a short enough time that anatomical movements have little or no effect on the imaging. In other situations, the acquisition time is longer and anatomical movements that occur negatively affect the imaging by, for example, distorting or obscuring the desired image.

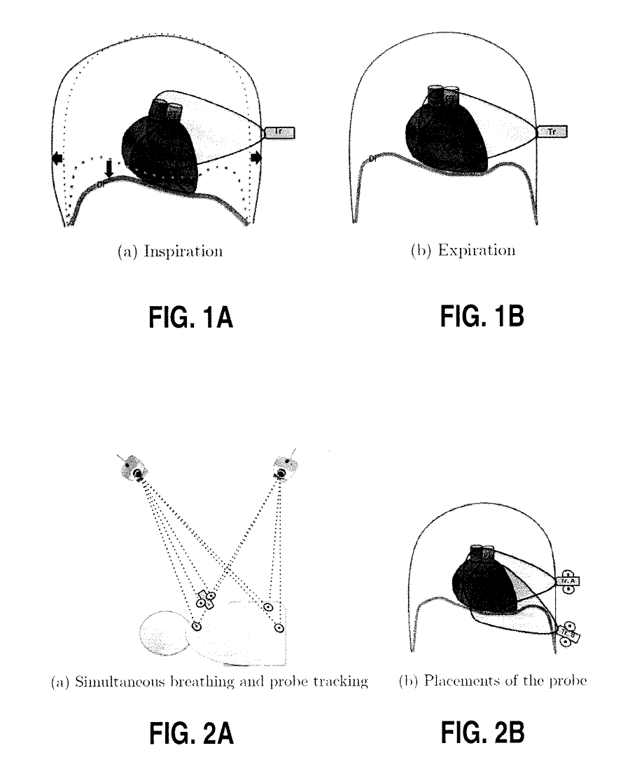

For example, movement of the heart due to breathing is an important aspect that affects the alignment of multiple scans. For a fixed position of the probe with respect to the patient, the position of the heart changes over the breathing cycle as depicted in FIGS. 1A-1B. To be suitable for fusion, the datasets need to be acquired when the heart is in the same position relative to the transducer or the movement of the heart should be compensated in the image alignment algorithm. Ignoring the heart movement due to the changes in the diaphragm may render the output of the fusion process useless.

The present disclosure is generally directed to an apparatus and method for generating a fused scan image from a plurality of anatomical scan images of a patient.

A tracking system is used to track the physical position and orientation of a scanner transducer, such as an ultrasound probe, which is used to obtain the anatomical scan images. The tracking system may also be used to track anatomical movement of the patient. The tracked position and orientation of the scanner transducer and the tracked patient anatomical movement may be used in the processing of the plurality of anatomical scan images for generating the fused scan image.

In some embodiments, the anatomical movement comprises respiratory movement of the patient.

The tracking allows the anatomical scanner to know the position and orientation of the scanner transducer when each of the plurality of anatomical scan images was captured. In addition, in some embodiments, the tracking allows the anatomical scanner to know or estimate movement of the patient's body due to respiratory movement when each of the plurality of anatomical scan images was captured. Movement of the patient's body during breathing may result in the movement of the organ, tissue, or bone being scanned. The tracking information may thus be used to generate more accurate or clearer fused images. In addition, the plurality of anatomical scan images may be processed and aligned using the tracked positional information without requiring any information of the images themselves for the alignment.

Furthermore, in some embodiments, an electrocardiogram (ECG) signal of a patient may be used in the process of generating the fused image. In an embodiment, tracking information generated by the tracking system may be time synchronized with the plurality of anatomical scan images based on the ECG signal. In an embodiment, an ECG signal may be used to identify and select only those anatomical scan images that were captured during a same phase of a heartbeat for generating the fused scan image. In this way, all of the scan images that are used were taken when the heart was in the same physical state. In an embodiment, the ECG signal may be used to identify and select only those anatomical scan images that were captured when the respiratory displacement of the patient was more or less the same. In this way, all of the scan images that are used were taken when chest of the patient was in the same physical position and state, which means that the heart and other organs in the chest were also in the same general physical location.

In various embodiments, the apparatus may comprise at least one of an optical tracking system, a mechanical tracking system, or an electromagnetic tracking system.

In an embodiment, the apparatus comprises a mechanical tracking system. A mechanical tracking system may comprise a measuring arm to obtain the instantaneous position and orientation of a scanner transducer, such as an ultrasound transducer, positioned at the distal end of the arm.

In an embodiment, the apparatus comprises an optical tracking system to align multiple ultrasound scans independent of any image information for alignment. A set of markers attached to the ultrasound transducer are tracked in 3D space by the multi-camera optical tracking system (see FIG. 2A). Another set of markers are placed on the chest and abdominal area of the subjects to estimate the respiratory motion and cycle. The example in FIG. 2B shows the movement of ultrasound probe during two different scans that can be combined to obtain a better field of view (FOV) than the individual scans. The transformations required to align multiple ultrasound scans were computed based on marker position.

In at least some embodiments, the present disclosure has one or more of the following advantages over previous image alignment approaches: (1) the image alignment does not suffer from any adverse image quality or artefacts due to speckle noise; (2) the accuracy of alignment is not constrained by the voxel resolution of the image; and (3) the movement of heart due to respiration is considered in the fusion process; and (4) it does not require an image overlap for alignment since it is independent of image information. The accuracy of alignment depends on the accuracy of optical tracking system which has a sub-millimeter precision, superior to a regular 3D ultrasound image resolution. In the method of the present disclosure, the markers are tracked using cameras, and therefore, it is not necessary to have a wired connection to the markers as in the case of electromagnetic tracking systems, which may constrain the ability to freely move the ultrasound transducer.

Another important aspect of the method of the present disclosure is the time-alignment of ultrasound scanning and tracking data. The typical time interval between two successive volumes in a cardiac 3D ultrasound acquisition is in the order of 10 milliseconds. Therefore, the time stamps provided by the ultrasound scanner and the tracking workstation are not reliable for synchronization. In order to synchronize, the method of the present disclosure uses an electrocardiogram (ECG) signal from the patient that was transmitted via the ultrasound scanner to the tracking workstation.

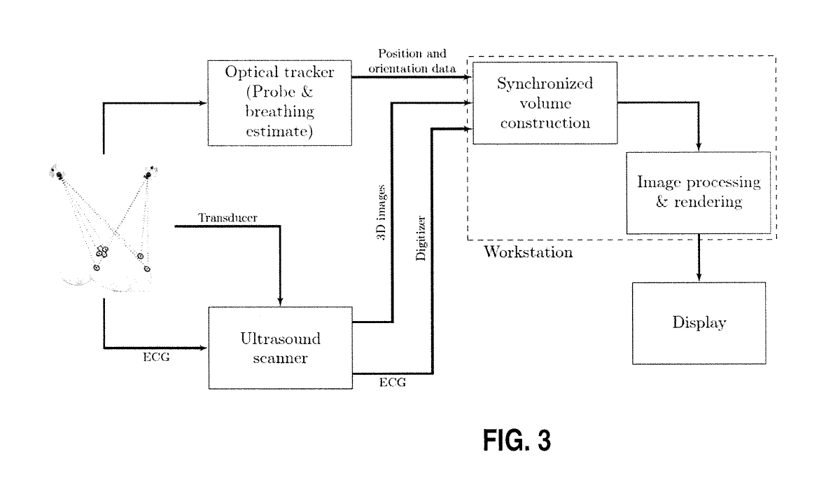

FIG. 3 shows the block diagram for the proposed system including an ultrasound scanner, an optical probe tracker, a workstation and a display. The ultrasound scanner receives the ECG signal and information from the ultrasound transducer and presents 3D images and a digitized ECG signal to the workstation. The optical probe tracker receives signals from the multi-camera optical tracking system (FIG. 2A) and generates position and orientation data based on the signals, which are delivered to the workstation.

The workstation includes one or more user input devices, and is configured for synchronized volume construction and image processing and rendering. The workstation receives inputs from the one or more input devices and provides an output to the display. The one or more input devices may include, for example, a mouse, keyboard, or digital interactive pen. The workstation communicates with and controls the ultrasound scanner and optical tracker. In some embodiments, the ultrasound scanner and optical tracker are located locally with the workstation. In other embodiments, the workstation communicates with and controls the ultrasound scanner and optical tracker through the internet, such as via a web-based application run on the workstation.

Although some embodiments are described as being implemented using a workstation, this is not meant to be limiting. Any other suitable computing devices may be used.

In addition to the FOV improvement, the fusion of multiple images has also been shown to improve the image quality and information such as the contrast, contrast-to-noise ratio, signal-to-noise ratio and anatomic features. These image improvements may lead to an increased reproducibility of echocardiography measurements. According to an aspect of the present disclosure, an image-processing based fusion technique is used to process a plurality of anatomical scan images to generate a fused scan image. In at least some embodiments, a wavelet-based fusion technique is employed to compute the fused image intensity values for the overlapping regions. The approach uses a pixel-wise likelihood estimate to assign weights to individual wavelet components, which ensures that pixel-wise information is optimized in the composite image. In at least some embodiments, a random walker fusion technique may be used to generate the fused scan image. In other embodiments, other suitable fusion techniques may be used, including but not limited to machine-learning based fusion techniques.

### Methodology

### Data

Three-dimensional data sequences were acquired on an ultrasound scanner using a matrix array transducer. Eighteen pairs of apical/parasternal image datasets were acquired from six healthy volunteers. The range of volume rate was 7-34 per cardiac cycle. The dimension of the volumes was 176×176×208 and the range of resolutions were (0.74×0.74×0.63)−(0.85×0.85×0.73) millimeters in x, y and z coordinate directions, respectively.

### Breathing Displacement Estimate

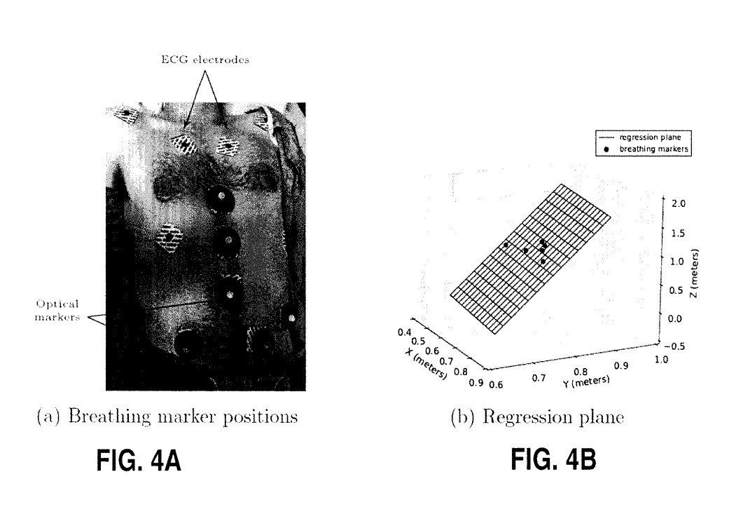

The markers attached to the chest and abdominal area of the subjects were tracked by the optical tracking system to estimate the respiratory movement. The displacement of the markers was estimated over the respiratory cycle by computing the normal distances to a regression plane estimated using the initial positions of all respiratory markers (see FIGS. 4A-B). The regression plane ax+by+cz+d=0 for the initial marker positions is computed as follows.

The normal vector v to the regression plane can be defined as

r=[a,b,c]^{T} (1)

and matrix M,

$$M = [ \begin{matrix}{x}_{1} - {x}_{0} & {y}_{1} - {y}_{0} & {z}_{1} - {z}_{0} \\ {x}_{2} - {x}_{0} & {y}_{2} - {y}_{0} & {z}_{2} - {z}_{0} \\ ⋮ & ⋮ & ⋮ \\ {x}_{m} - {x}_{0} & {y}_{m} - {y}_{0} & {z}_{m} - {z}_{0}\end{matrix} ] \qquad ( 2 )$$

where (xᵢ,yᵢ,zᵢ) is the position of ith marker (i=1, . . . ,m) and (x₀,y₀,z₀) is the centroid of the markers. The singular value decomposition (SVD) of M is given by

M=USV^{T} (3)

where S is a diagonal matrix containing the singular values of M, the columns of V are its singular vectors, and U is an orthogonal matrix. The regression plane contains the centroid (x₀,y₀,z₀) and its normal vector v is the singular vector of M corresponding to its smallest singular value. The proof thereof is now provided.

Let (xᵢ,yᵢ,zᵢ) be the position of the ith breathing marker. The linear regression plane ax+by+cz+d=0 can be found by minimizing

$$f ( a , b , c , d ) = {\sum }_{i = 1}^{m} \frac{{\vert {ax}_{i} + {by}_{i} + {cz}_{i} + d \vert}^{2}}{( {a}^{2} + {b}^{2} + {c}^{2} )}$$

Minimizing f(⋅) w.r.t. d,

$$\frac{\partial f}{\partial d} = 0$$

yields,

$$d = - {\sum }_{i = 1}^{m} \frac{( {ax}_{i} + {by}_{i} + {cz}_{i} )}{n} = - ( {ax}_{0} + {by}_{0} + {cz}_{0} )$$

Therefore the centroid (x₀,y₀,z₀) of the marker positions is on the regression plane. Substitute ford, we get

$$\begin{matrix}f ( v ) = {\sum }_{i = 1}^{m} \frac{{\vert a ( {x}_{i} - {x}_{0} ) + b ( {y}_{i} - {y}_{0} ) + c ( {z}_{i} - {z}_{0} ) \vert}^{2}}{( {a}^{2} + {b}^{2} + {c}^{2} )} \\ = \frac{{v}^{T} ( {M}^{T} M ) v}{{v}^{T} v}\end{matrix}$$

where v and Mare define above in equations (1) and (2), respectively. f(v) is a Rayleigh Quotient, which is minimized by the eigenvector of (M^{T}M) that corresponds to its smallest value.

Substitute SVD of M=USV^{T} after some algebraic manipulations, we get

M^{T}M=VS₂V^{T}

Therefore, it diagonalizes M^{T}M and provides an eigenvector decomposition. It means that the eigenvalues of M^{T}M are the squares of the singular values of M, and the eigenvectors of M^{7′} Mare the singular vectors of M.

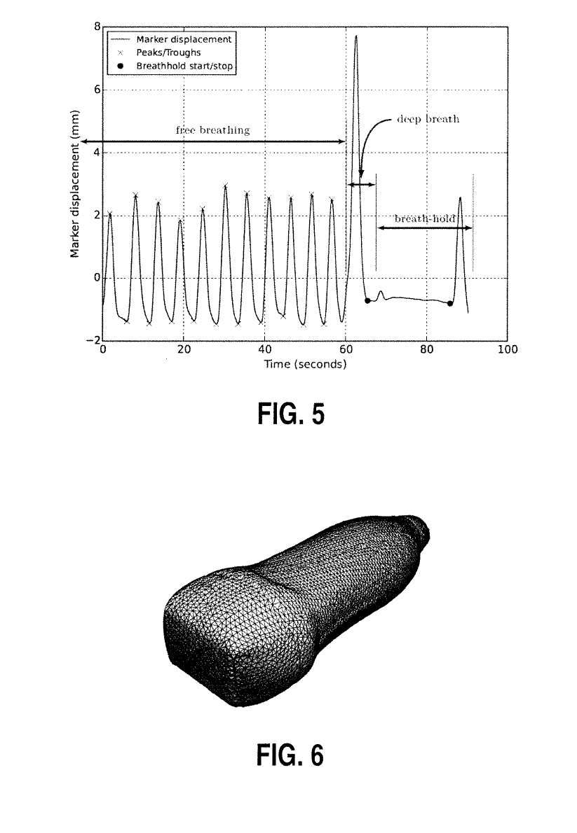

The average displacement over all markers at each time step was computed. A second order Butterworth filter was applied to smooth the data over time (see, e.g., FIG. 5).

Obtaining the Geometric Configuration of Markers Using a Laser Scanner

The markers attached to the transducer are tracked by the optical tracking system, and the position and orientation of the transducer is computed using these marker positions. Therefore, it is important to accurately estimate the geometry of the markers with respect to the transducer. In some embodiments, a laser scanner can be used to accurately obtain the geometric configuration of the markers with respect to the ultrasound transducer. This will allow computation of the geometric transformation, T_{probe}, associated with the marker positions, and the position and orientation of the ultrasound transducer. FIG. 6 shows the wireframe model of the echocardiography transducer obtained using the laser scanner.

### Optical Tracking System

Position and orientation of the transducer can be tracked using an optical tracking system (see FIG. 3). In some embodiments, the optical tracking system is a high precision tracking system that allows markers to be tracked down to sub-millimeter displacements. The method of the present disclosure allows six degrees of freedom of translational and rotational components when placing the transducer. The geometric transformation, T_{marker,n}, can be computed based on the positions of markers obtained from the optical tracking system for n^{th} scan as follows.

Let P={p₁,p₂, . . . ,pₗ} be the set of marker points obtained using a laser scan and Q={q_{1,n},q_{2,n}, . . . q_{l,n}} be the set of marker points obtained using the optical tracking system at scan n. Computation of the transformation can be formulated as the following optimization problem, which can be solved using a least square approximation.

$${T}_{marker , n} = {arg min}_{Φ} {\sum }_{j = 1}^{l} ‖ Φ {q}_{j , n} - {p}_{j} ‖ \qquad ( 1 )$$

### Geometric Transformation and Fusion of Ultrasound Images

The geometric transformation matrix, T_{total,n}, that transforms the ultrasound image acquired on n^{th} scan to a common coordinate system is computed by:

T_{total,n}=T_{probe}T_{marker,n} (5)

In some embodiments, the proposed algorithm can be implemented using the Python programming language and Visualization Toolkit.

### Synchronization of Tracking and Ultrasound Scanning

The ECG signal can be used to achieve the synchronization between the tracking system and ultrasound scanner. The echocardiography acquisition is generally performed between multiple R-R wave intervals. In some embodiments, the ECG signal is relayed through the ultrasound scanner and read using a digitizer from the computer. Average positional values over the acquisition interval were used in the computations.

### Wavelet-Based Image Fusion

The simplest approach to combine multiple images is to take the average intensity values of overlapping pixels. However, this may lead to deterioration in image quality since all views are equally weighted regardless of their individual image quality. In echocardiography, different areas of the heart are better captured by some views, and less well captured by others. When images from suboptimal views are averaged with images taken from optimal views, there can be a reduction in image quality. An alternative approach is to use a max norm where the pixel intensity of the composite image is determined as the maximum pixel intensity in any of the views. Although it might be a good option for high quality images this approach tends to increase noise levels in the composite image.

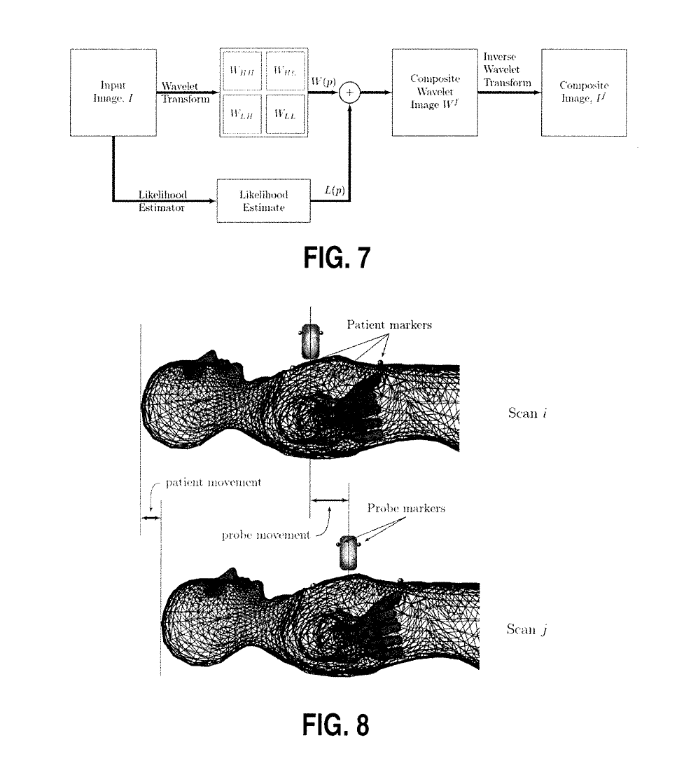

An embodiment according to the present disclosure addresses the shortcomings of other solutions by using a wavelet based fusion approach. An overview of the framework is shown in FIG. 7. The wavelet transform decomposes the input image into high and low frequency sub bands. For a two dimensional image it can be seen as cascaded high pass and low pass filtering in the horizontal and vertical dimensions resulting in four wavelet components W_{LL}, W_{HL} and W_{HH}. The low pass component W_{LL} is essentially a smoothed version of the input image while the high pass components correspond to horizontal (W_{HL}), vertical (W_{LH}) and diagonal (W_{HH}) edges.

The conventional reconstruction approach using wavelets would be to use a max norm for the high frequency sub-images and to average the low frequency sub-images. Since ultrasound images do not contain high frequency details, this results in blurred composite images. One approach is to use an inverse technique of maximizing the low frequency sub-images while averaging the high frequency sub-images. Although it solves the issues of blurring, the composite image is still susceptible to the aforementioned issues of noise enhancement and averaging over suboptimal images. The method of the present disclosure uses a pixel-intensity based likelihood estimator to address these issues.

The wavelet based reconstruction frame work can be formulated as follows. For a set of images I=I₁,I₂, . . . , I_{N}, let W=W₁, W₂, . . . , W_{N} represent the corresponding wavelet coefficients.

W=(W_{L,k},W_{H,k}) (6)

where W_{L,k} and W_{H,k} represent the low and high frequency sub-images respectively.

The wavelet components obtained from N views can be combined as follows:

$$\begin{matrix}{W}_{L} ( p ) = {\sum }_{k = 0}^{N} {W}_{L , k} ( p ) τ ( {l}_{k} ( p ) ) & ( 7 ) \\ {W}_{H} ( p ) = {\sum }_{k = 0}^{N} {W}_{H , k} ( p ) τ ( {l}_{k} ( p ) ) & ( 8 )\end{matrix}$$

where W_{L}(p) and W_{H}(p) represent the low and high frequency sub-images of the composite image respectively and Iₖ(p) represents the likelihood estimate for pixel p. The likelihood estimate Iₖ(p) is computed as follows:

$${l}_{k} ( p ) = exp ( - \frac{μ ( p )}{{σ}^{2} ( p ) {L}_{k}} ) \qquad ( 9 )$$

where μ(p) and σ²(p) represent the mean and standard deviation in the M pixel neighborhood of pixel p. These values can be computed as follows:

$$\begin{matrix}μ ( p ) = \frac{1}{M} {\sum }_{j = 0}^{M} {I}_{k} ( j ) & ( 10 ) \\ {σ}^{2} ( p ) = {\sum }_{j = 0}^{M} {( μ ( p ) - {I}_{k} ( j ) )}^{2} & ( 11 )\end{matrix}$$

The constant Lₖ is defined as the gray-level threshold of the image Iₖ. The value of Lₖ can be calculated using Otsu's method, which maximizes the interclass variance. The threshold operator r is defined as follows:

$$τ ( k ) = \{ \begin{matrix}1 & for k > {k}_{th} \\ 0 & for k \leq {k}_{th}\end{matrix} \qquad ( 12 )$$

The value of kₜₕ was set to the Otsu-threshold of the likelihood map Iₖ(p).

Finally, the composite image I^{f} from the fused wavelet components W_{L} and W_{H} can be obtained by

I^{f}=W^{I}(W_{L},W_{H}) (13)

where W^{I} is the inverse wavelet transform.

### Random Walker Based Fusion

As described above, an embodiment of the present disclosure uses a wavelet based image fusion approach. Another embodiment according to the present disclosure uses an image fusion approach that is based on a generalized random walker framework (GRW).

The GRW approach formulates fusion as a multi-labeling problem. For a set of n images coming from multiple views M={I₁, . . . , Iₙ} and set of labels L={I₁, . . . Iₙ} corresponding to these views, Random Walker (RW) algorithm finds the probability p of each pixel in the fused image having a label l∈L. The pixel intensity gⱼ^{f} can be calculated as the weighted average of the of the pixel intensities from the individual views.

$${g}_{i}^{f} = \frac{1}{n} {\sum }_{k = 1}^{n} {g}_{i} \times {p}_{i}^{k} \qquad ( 14 )$$

The set of pixels in fused image F and corresponding labels L are represented by nodes on an undirected graph G=(V, E) where V=(F∪L) and E=(F×L). The RW formulation finds the probability that a random walker starting from an image node v_{f}∈F reaches a particular label node v₁∈L. The edge weights for the image edges and label edges are represented by ωᵤ defined as:

$${ω}_{ij} = \{ \begin{matrix}exp ( - ( {g}_{i} - {g}_{j} ) ) & ∀ j ∈ F \\ exp ( - ( 1 - {U}_{i} ) ) & ∀ j ∈ L\end{matrix} \qquad ( 15 )$$

Where Uᵢ is the pixel probability of pixel i obtained from the Ultrasound Confidence Map (UCM) which gives a pixel-wise likelihood estimate ranging from 0 to 1 based on the location and neighborhood information of the pixel. We define UCM, Uᵢ, as follows:

Uᵢ=(dᵢ^{f}+dᵢ^{a})exp(−αdᵢ^{s})exp(−βFᵢ) (16)

Where dᵢ^{k} represents the distance between points i and k which is defined using a L² norm such that:

dᵢ^{k}=∥i−k∥² (17)

Fᵢ is a vesselness function computed based on eigen value decomposition Frangi et al (1998). Using eigen values (λ₁, λ₂) of the Hessian matrix H we define Fᵢ as:

$${F}_{i} = \{ \begin{matrix}0 & if {λ}_{2} > 0 \\ 1 - {e}^{\frac{{λ}_{1}}{{λ}_{2}}} + {e}^{( {λ}_{1} + {λ}_{2} )} & if {λ}_{2} \leq 0\end{matrix} \qquad ( 18 )$$

The Hessian matrix is computed as the convolution of the image I over the second order derivatives of a Gaussian filter bank G which can be written as:

$$G ( x , s ) = \frac{1}{2 π} {s}^{2} exp ( \frac{- {‖ x ‖}^{2}}{2 {s}^{2}} ) \qquad ( 19 )$$

The term s represents the scale of the Gaussian filter and was empirically set to 2. Similarly the two free parameters −α and β were empirically chosen for the entire dataset.

Based on the equivalence of random walker formulation and electrical networks we denote the node potential of vᵢ∈V as u(vᵢ). The total energy of the network can then be described in terms of a quadratic functional of the edge weights as:

$$E = \frac{1}{2} {\sum }_{( {v}_{i} , {v}_{j} )} {{ω}_{ij} ( u ( {v}_{i} ) - u ( {v}_{j} ) )}^{2} \qquad ( 20 )$$

This harmonic function can be efficiently computed using the Laplacian matrix L which represents the edge weights as:

$${L}_{ij} = \{ \begin{matrix}{d}_{i} & if i = j \\ {ω}_{ij} & if ( i , j ) ∈ V \\ 0 & otherwise\end{matrix} \qquad ( 21 )$$

The Laplacian matrix L can be rearranged using upper triangular matrices—L_{L}, L_{X} and R as:

$$L = [ \begin{matrix}{L}_{L} & R \\ R & {L}_{X}\end{matrix} ] \qquad ( 22 )$$

The energy functional in equation (15) can be solved as:

L_{X}uₓ=−R^{T}u_{L} (23)

The estimated contribution pᵢ^{k} of an individual view k for a pixel location i can be found by solving k such combinatorial formulations.

### Patient Movement Compensation

In an embodiment, the patient movement compensation is computed as follows. The movement of the patient between any two scans can be tracked using the markers placed on abdomen/chest of the patient (see FIG. 8). Let Tₚₐₜᵢₑₙₜ be a 4×4 transformation matrix associated with the patient movement. Tₚₐₜᵢₑₙₜ can be computed as described above. In order to reduce the effect of breathing, the average marker position over a period of time such as a breathing cycle will be used for computing Tₚₐₜᵢₑₙₜ. Let T_{probe} be the transformation associated with the probe movement between scans i and j. The relative transformation of the probe with respect to the patient is computed as shown above in equation (5).

Tᵣₑₗ instead of T_{probe} can be used in the fusion algorithm to obtain image alignment with patient movement compensation.

To validate the fusion with the patient movement compensation algorithm, a dynamic heart phantom was used. 3D echocardiography data sequences (dimension 176×208×224) were obtained at different probe locations using an ultrasound scanner at a volume rate of 20 Hz. The positions of the heart phantom between difference scans were also changed to mimic the patient movement. Prior to the experiment the positions of the probe markers were obtained using a laser scan. Optical markers placed on the probe as well as the phantom were tracked using an optical tracking system.

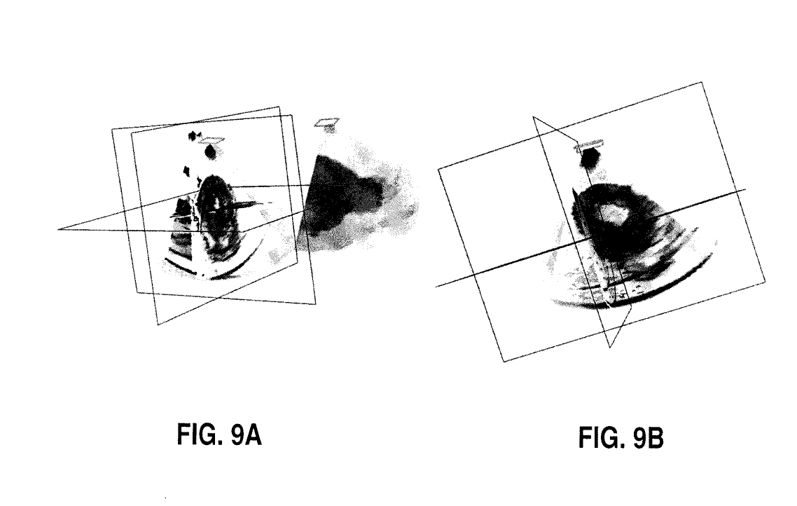

3D echocardiography sequences obtained at different positions are shown in FIG. 9A and the corresponding fused image is shown in FIG. 9B. As shown in FIG. 9B the fused images had clear myocardial borders despite being obtained at large spatial separation between the phantom locations.

Echocardiography Fusion with Free Breathing

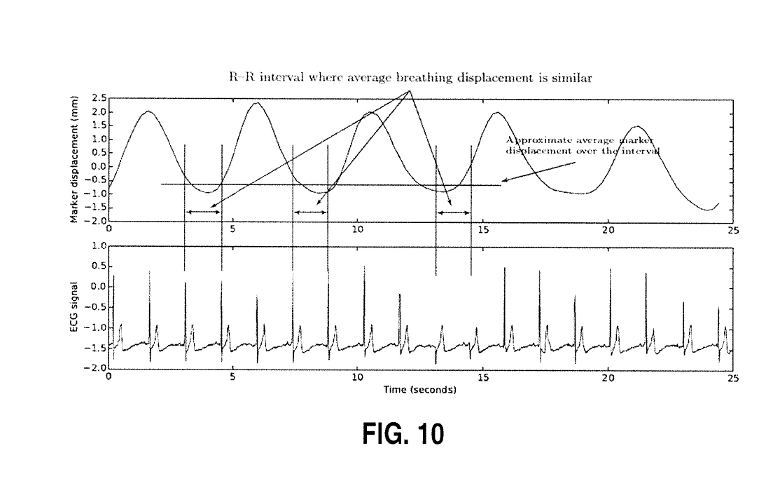

In the case of fusion with free breathing, the ultrasound data sets will be acquired continuously. The algorithm of the present disclosure selects the data sets to be fused based on breathing motion estimate. As depicted in FIG. 10, the algorithm will compute average and variance of breathing marker displacement for each R-R interval. The data sets which have more or less the same average values for the displacement will be fused. A predefined threshold will be used to decide the acceptable variations in average displacement values over the R-R interval. The variance (or the difference between the largest and smallest displacement within the R-R interval) of the marker displacement will be used to infer the amount of the heart movement within the R-R interval, and data sets that correspond to larger marker displacement within the R-R interval will be discarded.

### Experimental Results

### Visual Inspection

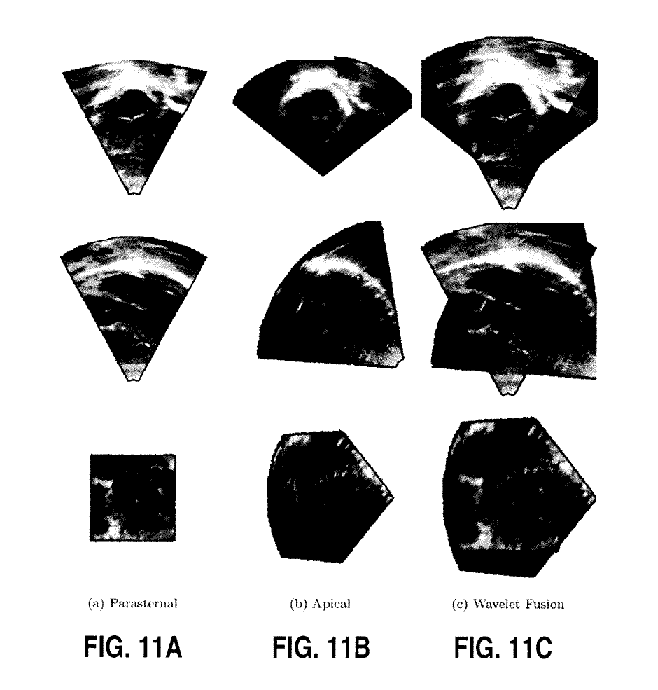

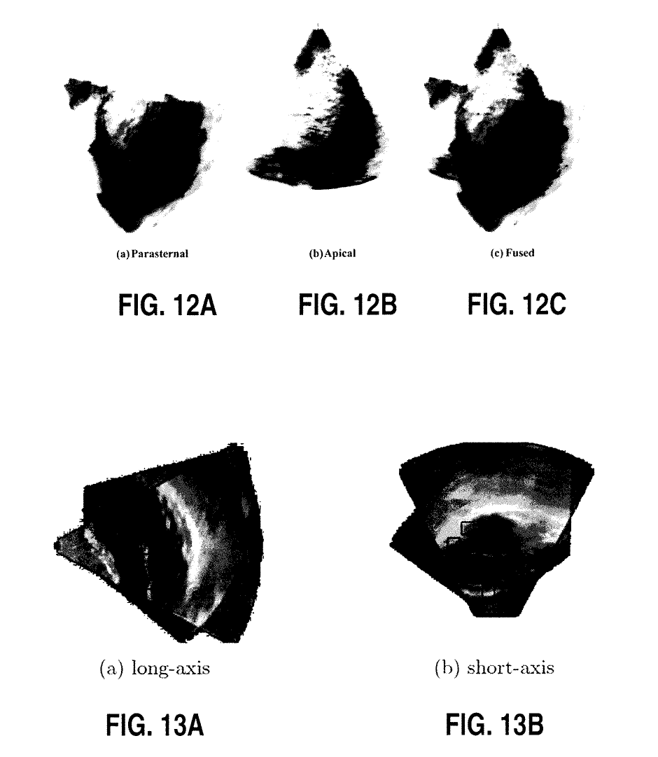

The alignment of multiple scans were visually assessed as shown in the example in FIGS. 11A-C. The visual inspection was performed by animating the sequence of image volumes and assessing the alignment using three different orthogonal planes. A 3D volume rendered animation was also used over the entire cardiac cycle for both parasternal and apical views in order to assess the alignment accuracy (see FIGS. 12A-C for an example screencast). The method of the present disclosure provided excellent alignment of parasternal and apical echocardiography scans for both single breath-hold and subsequent breath-hold acquisitions regardless of the image quality.

The proposed algorithm took an average of 0.076±0.012 seconds on a 3.50 GHz CPU to compute the transformation for a pair of volumes estimated over 267 volume pairs.

Quantitative Analysis Using Fusion Quality Metrics

For quantitative evaluation three square regions (of 10×10 pixels each) were manually selected in the Myocardial (MY) and Bloodpool (BP) regions. The gray-scale values for pixels inside the regions was obtained using MATLAB. The following fusion quality metrics were used to quantitatively compare the results of the fusion process with the original images.

The percentage improvement in contrast indicates the difference in mean intensity between the myocardial and blood pool regions, which is calculated as follows:

$$Δ Contrast = [ \frac{{μ}_{f , MY} - {μ}_{f , BP}}{\frac{1}{N} {\sum }_{k = 0}^{N} ( {μ}_{k , MY} - {μ}_{k , BP} )} - 1 ] \times 100 \qquad ( 24 )$$

where μ_{f,MY} and μ_{f,BP} represent mean intensities in myocardial and blood pool regions of the fused image.

The contrast-to-noise ratio (CNR) is computed as follows:

$$Δ CNR = [ \frac{\frac{{μ}_{f , MY} - {μ}_{f , BP}}{{σ}_{f , MY}^{2} - {σ}_{f , BP}^{2}}}{\frac{1}{N} {\sum }_{k = 0}^{N} \frac{{μ}_{k , MY} - {μ}_{k , BP}}{{σ}_{k , MY}^{2} + {σ}_{k , BP}^{2}} - 1} ] \times 100 \qquad ( 25 )$$

Signal-to-noise ratio (SNR) is the ratio of image intensity to noise. The overall SNR improvement SNR was calculated as the average of SNR improvements in the myocardial SNR_{MY} and blood pool regions SNR_{BP} (Refer to FIGS. 13A-B for an example). This can be calculated as follows:

$$\begin{matrix}Δ \overline{SNR} = \frac{Δ {SNR}_{MY} + Δ {SNR}_{BP}}{2} & ( 26 ) \\ Δ SNR = [ \frac{{SNR}_{f}^{+}}{\frac{1}{N} {\sum }_{k = 0}^{N} {SNR}_{k}^{+}} - 1 ] \times 100 & ( 27 ) \\ S N {R}_{k}^{+} = \frac{{μ}_{k}}{{σ}_{k}} & ( 28 )\end{matrix}$$

where μₖ represent the mean intensity and σₖ represents the variance in the region k.

A number of Gabor features extracted from the image were used to compute an image quality metric. In 2D domain the Gabor filter can be seen as a Gaussian function modulated by a sinusoidal plane wave. The value of the pixel at a location (x,y) can be calculated as follows:

$$\begin{matrix}G ( x , y ) = \frac{{f}^{2}}{π γ η} exp ( - \frac{{\tilde{x}}^{2} + {γ}^{2} {\tilde{y}}^{2}}{2 {σ}^{2}} ) exp ( j 2 π f \tilde{x} + σ ) & ( 29 ) \\ where & \\ \tilde{x} = x cos θ + y sin θ & ( 30 ) \\ \tilde{y} = - x sin θ + y cos θ & ( 31 )\end{matrix}$$

The symbols f represents frequency, Θ represents orientation, φ represents phase offset, a represents standard deviation and, y and n represent the ratio of frequency to sharpness of the Gabor function along the major and minor axis, respectively. The following parameter values for Gabor filtering were used: f=0.25; and γ=η=\sqrt{2}.

Based on the Gabor function the feature count improvement ΔFC can be expressed as follows:

$$Δ FC = [ \frac{{FC}_{f}}{\frac{1}{N} {\sum }_{k = 1}^{N} {FC}_{k}} - 1 ] \times 100 \qquad ( 32 )$$

where FC is the number of the significant Gabor features in the image. The algorithm used calculates the Gabor filter outputs of the image in five scales and eight scales. During the experiments all features above a threshold value of 0.1 were considered to be significant.

Field of view (FOV) was defined as the number of pixels inside the ultrasound volume. This can be mathematically expressed as follows:

$$\begin{matrix}Δ F O V = [ \frac{F O {V}_{f}}{\frac{1}{N} {\sum }_{k = 0}^{N} F O {V}_{k}} - 1 ] \times 100 & ( 33 ) \\ F O V = {\sum }_{k = 0}^{n} r ( i ) & ( 34 ) \\ r ( i ) = \{ \begin{matrix}1 , & r ( i ) ∈ V \\ 0 , & otherwise\end{matrix} & ( 35 )\end{matrix}$$

where V represents the set of pixels in the ultrasound volume.

### Qualitative Study

A qualitative study was conducted to evaluate various clinically relevant parameters from the image: 1) clarity of myocardial border; 2) noise level of the image; 3) contrast of the image; 4) sharpness of the image; and 5) clarity of leaflet (if present). An image test-set comprising of parasternal, apical and fused views were presented to each evaluator in random order. The images were cropped such that only the overlapping region was visible so as to avoid any disquisition between fused or single image. The images were scored on a scale of 1-4 with 4 being the highest score indicating the best quality.

A 22% improvement in SNR was observed while using average fusion. Maximum fusion increased the contrast of the image (42%). An ANOVA test was conducted to determine the statistical significance of the results. The wavelet based fusion technique of the present disclosure gave a mean contrast improvement of 24% greater than that of max fusion. The average SNR of wavelet fusion was 35% (Average vs Wavelet, p<0.001) greater than that of average fusion and 56% (Maximum vs Wavelet, p<0.001) greater than max fusion. The contrast to noise ratio (CNR) of the proposed approach was 25% (Average vs Wavelet, p<0.001) greater than average fusion and 27% (Maximum vs Wavelet, p<0.001) greater than max fusion. The wavelet based fusion described in Rajpoot K, Noble J A, Grau V, Szmigielski C, Becher H. Multiview RT3D echocardiography image fusion. In: Functional Imaging and Modeling of the Heart. Springer, 2009. pp. 134-143 (Rajpoot et al.) which showed improvements of 41%, 30% and 9% for contrast, CNR and SNR respectively.

The average feature count was increased by 13% by wavelet method which was 5% (Average vs Wavelet, p=0.90), higher than that of average fusion and 6% (Maximum vs Wavelet, p=0.31) higher than max fusion. The results of quantitative evaluation of fusion techniques is summarized in Table 1 below. Upon visual assessment the composite image obtained from wavelet fusion displayed more contrast near the myocardial boundary and reduced speckle noise inside the blood pool region.

TABLE 1
The percentage improvements of the image quality metrics for
different fusion approaches.

| Method | ΔContrast | ΔCNR | ΔSNR | ΔFC |
|---|---|---|---|---|
| Average (AVG) | 0 | 24.04 ± 12.18 | 22.50 ± 11.19 | 8.25 ± 13.21 |
| Maximum (MAX) | 42.19 ± 25.02 | 21.73 ± 16.64 | 1.19 ± 11.06 | 7.71 ± 12.32 |
| Rajpoot et al.(WAV) | 40.93 ± 25.66 | 30.18 ± 18.93 | 9.02 ± 12.02 | 10.45 ± 11.04 |
| Our method (WAVL) | 66.46 ± 21.68 | 49.92 ± 28.71 | 57.59 ± 47.85 | 13.06 ± 7.44 |
| WAVL cs. AVG p-value | <0.001 | <0.001 | <0.001 | 0.90 |
| WAVL cs. MAX p-value | <0.001 | <0.001 | <0.001 | 0.31 |
| WAVL cs. WAV p-value | <0.001 | <0.001 | <0.001 | 0.92 |

CNR = Contrast to Noise Ratio,
SNR = Signal to Noise Ratio,
FC = Feature Count.

The inter-observer variability in the qualitative scores for the matrices were: 0.6±0.7 for myocardial border, 0.5±0.5 for noise level, 0.3±0.4 for contrast, 0.5±0.3 for sharpness, and 0.4±0.2 for leaflet. The fusion technique of the present disclosure showed an improvement of 35% in FOV. The improvement in FOV was considerably higher than corresponding values reported in Rajpoot et al. In contrast to the method in Rajpoot et al., the method of the present disclosure does not rely on image information for alignment, and therefore, it is possible to acquire scans that are far apart. The fused image provided was able to capture most of the geometry of the heart. This was useful in visualizing boundary features that were not completely visible in a single ultrasound view.

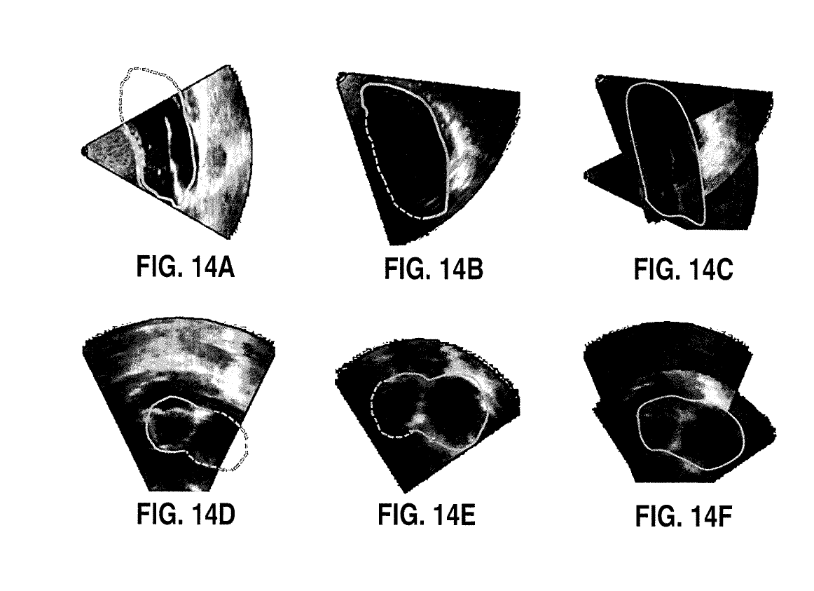

FIGS. 14A-F shows a representative example of single and composite echocardiography images. Images FIGS. 14A, 14B, 14D and 14E show the individual views obtained from different scanning locations while images FIGS. 14C and 14F show the corresponding the composite images. It can be seen that the left ventricular (LV) myocardial border is clearly visible in image FIG. 14F as opposed to single views FIG. 14D and FIG. 14E where the myocardial borders are not clearly visible. The results of the qualitative evaluation of images in comparison to the individual parasternal and apical views is summarized in Table 2 below.

TABLE 2
The results of qualitative evaluation on scale of 1-4.

| | Myocardial | Noise | | | Leaflet |
|---|---|---|---|---|---|
| View | border | level | Contrast | Sharpness | clarity |
| Parasternal (PAR) | 3.10 ± 1.15 | 3.16 ± 0.91 | 3.13 ± 1.04 | 2.96 ± 1.18 | 3.14 ± 1.24 |
| Apical (API) | 2.87 ± 0.89 | 3.03 ± 0.80 | 2.76 ± 1.04 | 2.70 ± 1.05 | 2.86 ± 1.15 |
| Our method (WAVL) | 3.33 ± 0.88 | 3.20 ± 0.92 | 3.43 ± 0.89 | 3.20 ± 1.03 | 3.48 ± 1.03 |
| WAVL cs. API p-value | 0.03* | 0.43 | 0.01* | 0.04* | 0.13 |
| WAVL cs. PAR p-value | 0.41 | 0.88 | 0.18 | 0.44 | 0.38 |

The wavelet-fusion algorithm was implemented in MATLAB. The execution time of the image fusion algorithm averaged over 242 images was 0.172±0.047 seconds on a 2.30 GHz CPU.

### Mechanical Tracking System

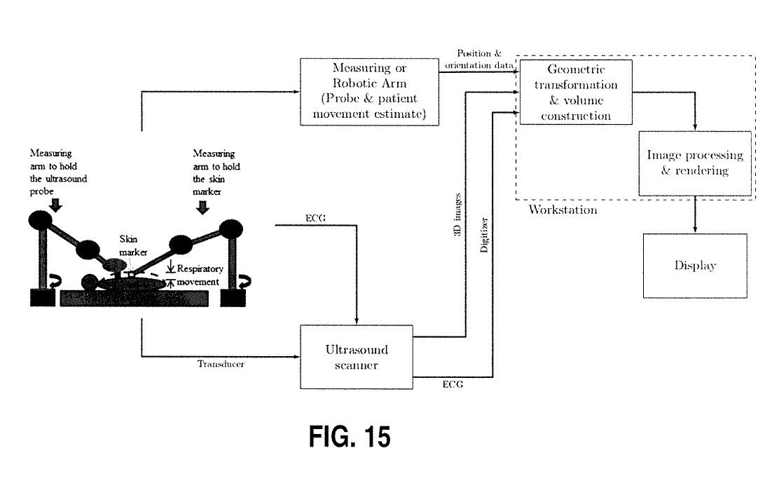

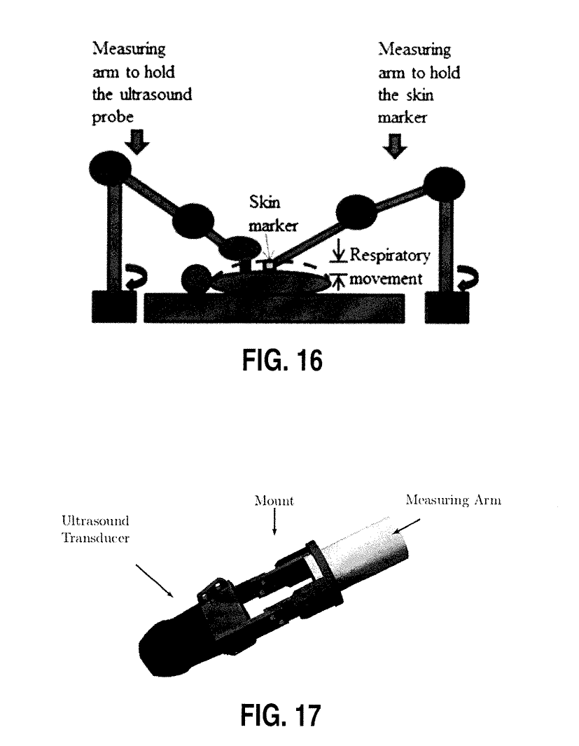

Referring to FIG. 15, in another embodiment, position and orientation of the scanner transducer may be tracked using a mechanical tracking system. FIG. 16 is a close-up view of the mechanical tracking system of FIG. 15. A mechanical tracking system may comprise a measuring arm to obtain the instantaneous position and orientation of a scanner transducer, such as an ultrasound transducer, positioned at the distal end of the arm. Sensors in the arm may be used to track the instantaneous positions and orientations of the end of the arm. The arm may produce one or more output signals that may be communicated to the anatomical scanner. In an embodiment, the mechanical tracking system may comprise a measuring arm configured for tracking the instantaneous position, and in some embodiments the orientation, of a skin marker positioned at the patient for tracking respiratory movement or other anatomical movement of the patient. In an embodiment, the tracking system comprises two measuring arms for tracking the scanner transducer position/orientation and anatomical movement.

The scanner transducer may be attached to a distal end of a measuring arm using any suitable mount or other attachment means. A second measuring arm may be employed for tracking the respiration and patient movement during the image scanning. The second arm may be attached to skin marker on the patient. The image scanning apparatus may use the information from the respiratory and patient movement tracking to compensate for any resulting misalignment of the image scans. The measuring arm may have sufficient degrees of freedom to allow the attached scanner transducer to move freely.

FIG. 17 shows a distal end of example measuring arm and a mount extending therefrom for securing a scanner transducer.

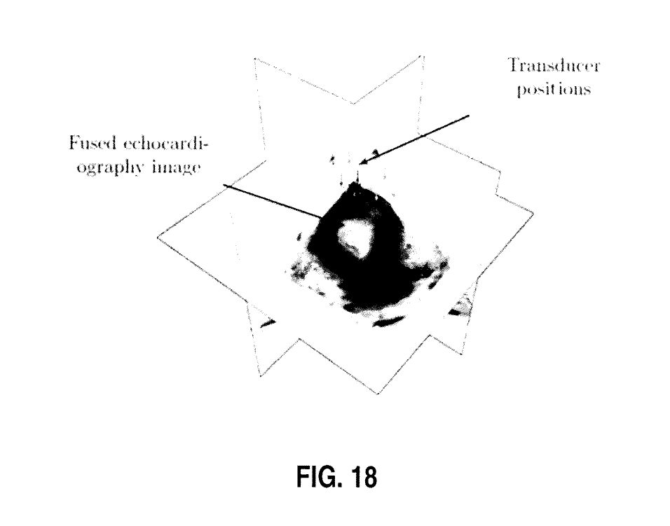

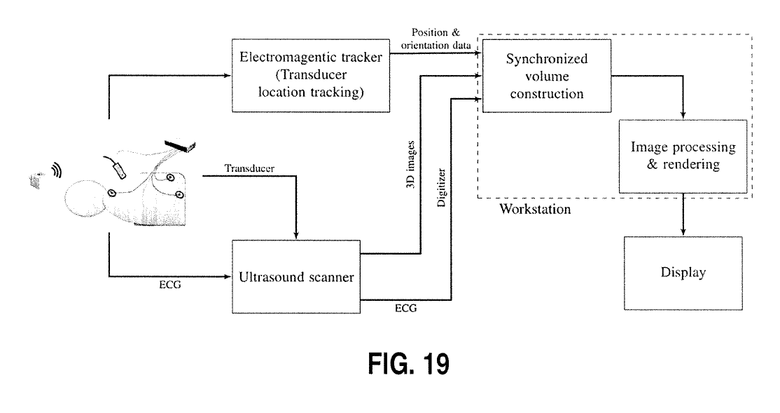

FIG. 18 is an example 3 dimensional image showing the fusion of multiple echocardiography scans where the transducer placements were tracked using a measuring arm. In this example, three-dimensional echocardiography datasets of a dynamic heart phantom (Shelly Medical Technologies, London, Ontario, Canada) were acquired using a Siemens ACUSON SC2000 scanner (Siemens Healthcare, Erlangen, Germany). Siemens Volume Viewer software was used to export the scans to Cartesian coordinate system. The dimension of the Cartesian data set is 198×187×172 and the voxel spacing is 1 mm in all x, y and z-coordinate directions.

The location of the transducer was obtained using a measuring arm (Faro Technologies, Lake Mary, Fla., United States). A custom-designed mount was used to attach the transducer to the measuring arm (see FIG. 17). The outer surface of the transducer was obtained using a laser scanner (Kreon Technologies, Limoges, France) and used in designing the mount using OpenSCAD, an open-source 3D modeling software. The relative transformation between the measuring arm and the scanner transducer was computed based on the design of the mount. The mount was printed using 3D printing technology.

The fusion system was implemented in Python programming language. The transformation computations were performed on an Intel Core i7 processor with 16 GB RAM. The results were rendered using NVIDIA GeForce GTX 1060 graphics card.

Nine single echocardiography scans were acquired with small transducer displacements. The arrows in FIG. 18 indicate the locations and directions of the transducer placements in 3D space. FIG. 18 shows the fused single dataset of all nine scans. The visual assessment of the fused data set demonstrated that the measuring arm can be used for accurately track the location of the transducer positions and orientations in place of optical or electromagnetic tracking systems for the fusion technology.

### Electromagnetic Tracking System

Referring to FIG. 19, in another embodiment, position and orientation of the scanner transducer may be tracked using an electromagnetic tracking system. An electromagnetic tracking system generally comprises a transmitter and a plurality of electromagnetic sensors, and the systems utilizes the transmitter to localize the electromagnetic sensors in an electromagnetic field of known geometry. The electromagnetic tracking system may be configured to provide signals that may be used to determine the instantaneous position and orientation of a scanner transducer, such as an ultrasound transducer. The electromagnetic tracking system may produce one or more output signals that may be communicated to the anatomical scanner.

In an embodiment, the electromagnetic tracking system may comprise one or more electromagnetic sensors configured for tracking the respiratory movement or other anatomical movement of the patient. The one or more electromagnetic sensors may be used to determine the instantaneous position, and in some embodiments the orientation, of one or more skin markers positioned at the patient for tracking the respiratory movement or other anatomical movement. In an embodiment, the electromagnetic tracking system may be configured for tracking both the scanner transducer position/orientation and anatomical movement.

An electromagnetic tracking system generally does not suffer from a line-of-sight limitation of optical systems. Further, in some embodiments, an electromagnetic tracking system does not require an initial calibration to track the transducer in 3D space. By utilizing a plurality of electromagnetic sensors to track the transducer and using a laser scanner device to accurately determine the geometric configuration of the sensors relative to the scanner transducer, the electromagnetic tracking system may allow for the direct computation of transformations and may remove the need for initial calibration. In an embodiment, three electromagnetic sensors may be used to track the transducer scanner. In other embodiments, fewer or more sensors may be used. Further, in an embodiment, the electromagnetic sensors are miniaturized, which allows them to be seamlessly integrated with the scanner transducer.

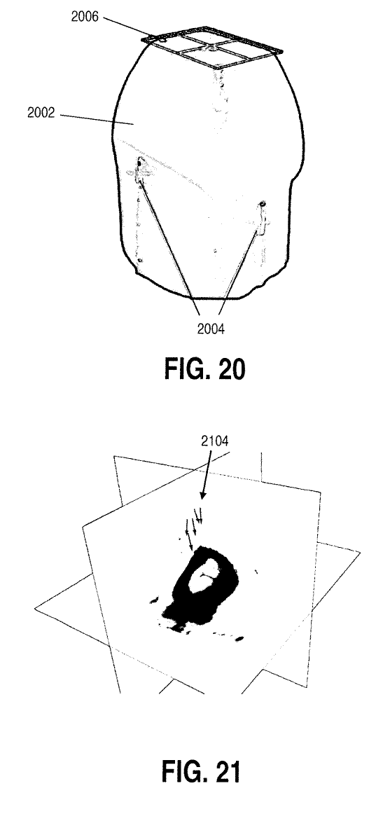

FIG. 20 is a surface representation of an ultrasound scanner transducer 2002 and electromagnetic sensors 2004 obtained using a laser scanner device. The laser scanner device was used to obtain accurate geometric configuration of the electromagnetic sensors relative to the scanner transducer. A transducer reference plane 2006 is also indicated in FIG. 20. Once the positions of the one or more sensors are determined relative to the scanner transducer, the electromagnetic tracking system may be used to track the position of the sensors during an anatomical scan.

The tracking may be computed and performed according to algorithms previously described. For example, a geometric transformation matrix may be computed based on the positions of sensors obtained from the electromagnetic tracking system for nth scan as follows. Let P={p₁, p₂, . . . , pₗ} be the set of sensor points obtained using the laser scanner and Q={q_{1,n}, q_{2,n}, . . . q_{l,n}} be the set of points obtained using the electromagnetic tracking system at scan n. Computation of the transformation may be formulated as the optimization problem represented by equation (4) above. This problem may be solved using a least square approximation.

An experiment was conducted using an electromagnetic tracking system according to the present disclosure. Electromagnetic tracking sensors were attached to an ultrasound transducer as shown in FIG. 20. A laser scanner (Kreon 3D scanner, Lemoges, France) was used to determine the locations of the electromagnetic sensors and the ultrasound transducer sensor array in order to compute the geometric transformation associated with the sensor configuration.

Three-dimensional ultrasound data sets were acquired on a Siemens ACUSON SC2000 scanner (Siemens Healthcare, Erlangen, Germany). Siemens Volume Viewer software was used to export the ultrasound data sets to Cartesian coordinate system. The dimension of the Cartesian data set was 196×187×172 and the voxel resolution was 1.0 mm in all x, y and z coordinate directions. A dynamic heart phantom (Shelley Medical Imaging Technologies, London, Ontario, Canada) was scanned by placing the ultrasound transducer at different locations. A trakSTAR electromagnetic system (Northern Digital Inc., Waterloo, Ontario, Canada) was used to obtain and track the location of the sensor positions.

An algorithm according to the present disclosure for processing the data was implemented using Python programming language with numpy module and Visualization Toolkit (VTK, Kitware, New York, USA). The fused output image volumes were rendered using an NVIDIA Quadro K5000 graphics card.

Five echocardiography scans with small transducer displacements were acquired. FIG. 21 is a 3 dimensional image showing the fusion of the multiple echocardiography scans where the transducer placements were tracked using an electromagnetic tracking system. The arrows in FIG. 21 indicate the location and direction of the transducer in 3D space. The scans include volume acquisition with rotated transducer positions.

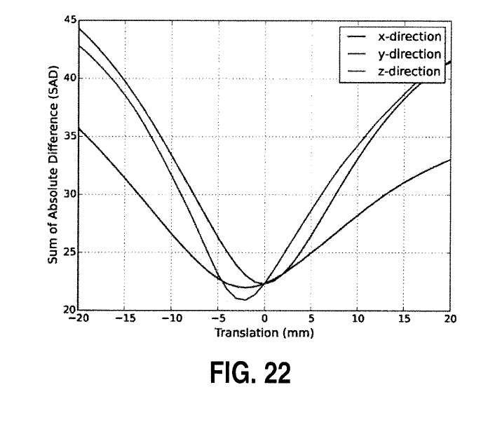

In order to assess the accuracy of alignment quantitatively, the sum of absolute difference (SAD) between two ultrasound image volumes were computed by artificially introducing translations in x, y and z coordinate directions from the obtained alignment position using the proposed method. The SAD between two image volumes Ω₁ and Ω₂ is computed as follows:

$$S A {D}_{{Ω}_{1} , {Ω}_{2}} = {\sum }_{u , v , w ∈ [ {Ω}_{1} ⋂ {Ω}_{2} ]} \vert {I}_{{Ω}_{1}} ( u , v , w ) - {I}_{{Ω}_{2}} ( u , v , w ) \vert \qquad ( 36 )$$

where IQ_{Ω1} (u, v, w) and I_{Ω2} (u, v, w) denote the image intensity values at point (u, v, w) for Ω₁ and Ω₂, respectively. [Ω₁∩Ω₂] denotes the overlapping region of Ω₁ and Ω₂. FIG. 22 is a representative example plot for SAD vs artificially introduced translation for an image volume pair. In particular, FIG. 22 is a representative example showing the sum of absolute difference (SAD) versus an artificially introduced translation in x, y, z coordinate directions from the obtained alignment using the method between two scans. The SAD was computed over the overlapping region of the two echocardiography volumes. The orientation of the image pairs used for this example were orthogonal to each other. The plot shows that the proposed method yielded an alignment closer to the optimal alignment in terms of SAD between the scans.

The method in the experiment provided a nearly optimal alignment in the fusion of multiple scans. The tracking may be improved to reduce the subsequent error in the computing the transformation. In an embodiment, the measurement error may be reduced by continuously tracking the sensor positions and applying recursive Bayesian filtering. In an embodiment, the orientation information provided by the electromagnetic tracker may be exploited in addition to the positional information to improve the tracking of the scanner transducer.

The method used in the experiment does not rely in any image information for alignment, and therefore, it may also be used in ultrasound applications where the signal-to-noise ratio is low. Also, the time taken by the method to find image alignment is much smaller than the typical time required by an image registration based approach which often involves in computationally expensive optimization to find the solution.

### Combination of Tracking Systems

In some embodiments, different types of tracking systems may be used in combination. In particular, in an embodiment, a system may comprise two or more of an optical tracking system, a mechanical tracking system, and an electromagnetic tracking system. For example, one of the tracking systems may be used to track the position and/or orientation of the scanner transducer, while another tracking system may be used to track anatomical movement of the patient.

### Process

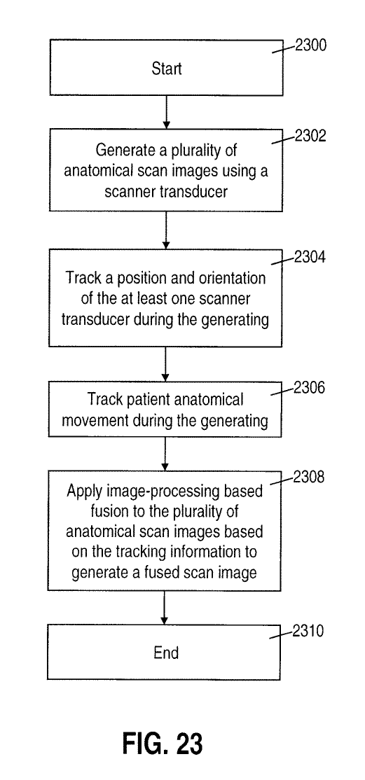

FIG. 23 shows a process for generating a fused scan image in an embodiment according to the present disclosure. The process starts at block 2300 and proceeds to block 2302, where a plurality of anatomical scan images of a patient are generated with a scanner transducer.

The process then proceeds to block 2304, where a position and orientation of the at least one scanner during the generating is tracked.

The process then proceeds to block 2306, where patient anatomical movement during the generating is tracked.

The process then proceeds to block 2308, where image-processing based fusion is applied to the plurality of anatomical scan images based on the tracked position and orientation of the at least one scanner transducer and the tracked patient anatomical movement to generate a fused scan image.

The process then proceeds to block 2310 and ends.

Although various embodiments have been described for anatomical imaging in the form of echocardiography, this is not meant to be limiting. The present disclosure may be used in other types of imaging including forms of anatomical imaging other than echocardiography. Furthermore, the present disclosure is not limited to ultrasound imaging; it may be used in other types of medical or anatomical imaging.

### Electronic Device

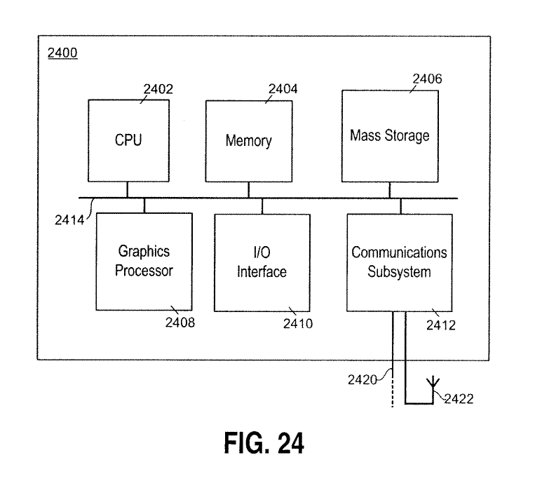

FIG. 24 is a block diagram of an example electronic device 2400 that may be used in implementing one or more aspects or components of an embodiment according to the present disclosure. As previously described, in an embodiment, the scanning apparatus may comprise a work station.

The electronic device 2400 may include one or more of a central processing unit (CPU) 2402, memory 2404, a mass storage device 2406, an input/output (I/O) interface 2410, a communications subsystem 2412, and a graphics processor 2408. One or more of the components or subsystems of electronic device 2400 may be interconnected by way of one or more buses 2414 or in any other suitable manner.

The bus 2414 may be one or more of any type of several bus architectures including a memory bus, storage bus, memory controller bus, peripheral bus, or the like. The CPU 2402 may comprise any type of electronic data processor. The memory 2404 may comprise any type of system memory such as dynamic random access memory (DRAM), static random access memory (SRAM), synchronous DRAM (SDRAM), read-only memory (ROM), a combination thereof, or the like. In an embodiment, the memory may include ROM for use at boot-up, and DRAM for program and data storage for use while executing programs.

The mass storage device 2406 may comprise any type of storage device configured to store data, programs, and other information and to make the data, programs, and other information accessible via the bus 2414. The mass storage device 2406 may comprise one or more of a solid state drive, hard disk drive, a magnetic disk drive, an optical disk drive, or the like. In some embodiments, data, programs, or other information may be stored remotely, for example in the “cloud”. Electronic device 2400 may send or receive information to the remote storage in any suitable way, including via communications subsystem 2412 over a network or other data communication medium.

The graphics processor 2408 may be any suitable type of processor for processing graphics. In an embodiment, the graphics processor 2408 may be part of a graphics adapter or graphics card, which may comprise other components such as graphics memory and one or more output ports for interfacing with one or more video displays (not shown). As previously described, in some embodiments a graphics adapter may be an NVIDIA GeForce GTX 1060 graphics card or an NVIDIA Quadro K5000 graphics card, without limitation.

The I/O interface 2410 may provide interfaces to couple one or more other devices (not shown) to the electronic device 2400. The other devices may include but are not limited to one or more of an anatomical scanner, and one or more components of a tracking system such as a measuring arm, electromagnetic tracker, or camera. Furthermore, additional or fewer interfaces may be utilized. For example, one or more serial interfaces such as Universal Serial Bus (USB) (not shown) may be provided.

A communications subsystem 2412 may be provided for one or both of transmitting and receiving signals. Communications subsystems may include any component or collection of components for enabling communications over one or more wired and wireless interfaces. These interfaces may include but are not limited to USB, Ethernet, high-definition multimedia interface (HDMI), Firewire (e.g. IEEE 1394), Thunderbolt™, WiFi™ (e.g. IEEE 802.11), WiMAX (e.g. IEEE 802.16), Bluetooth™, or Near-field communications (NFC), as well as GPRS, UMTS, LTE, LTE-A, dedicated short range communication (DSRC), and IEEE 802.11. Communication subsystem 2412 may include one or more ports or other components 2420 for one or more wired connections. Additionally or alternatively, communication subsystem 2412 may include one or more transmitters (not shown), receivers (not shown), and/or antenna elements 2422.

The electronic device 2400 of FIG. 24 is merely an example and is not meant to be limiting. Various embodiments may utilize some or all of the components shown or described. Some embodiments may use other components not shown or described but known to persons skilled in the art.

In the preceding description, for purposes of explanation, numerous details are set forth in order to provide a thorough understanding of the embodiments. However, it will be apparent to one skilled in the art that these specific details are not required. In other instances, well-known computer and/or electrical related structures and circuits are shown in block diagram form in order not to obscure the understanding. For example, specific details are not necessarily provided as to whether the embodiments described herein are implemented in software, in hardware, firmware, or any combination thereof.

Embodiments or portions therefore in accordance with the present disclosure may be represented as a computer program product stored in a machine-readable medium (also referred to as a computer-readable medium, a processor-readable medium, or a computer usable medium having a computer-readable program code embodied therein). The machine-readable medium can be any suitable tangible, non-transitory medium, including magnetic, optical, or electrical storage medium including a diskette, compact disk read only memory (CD-ROM), memory device (volatile or non-volatile), or similar storage mechanism. The machine-readable medium can contain various sets of instructions, code sequences, configuration information, or other data, which, when executed, cause a processor to perform steps in a method according to an embodiment of the disclosure. Those of ordinary skill in the art will appreciate that other instructions and operations necessary to implement the described implementations can also be stored on the machine-readable medium. The instructions stored on the machine-readable medium can be executed by a processor or other suitable processing device, and can interface with circuitry to perform the described tasks.

The structure, features, accessories, and alternatives of specific embodiments described herein and shown in the Figures are intended to apply generally to all of the teachings of the present disclosure, including to all of the embodiments described and illustrated herein, insofar as they are compatible. In other words, the structure, features, accessories, and alternatives of a specific embodiment are not intended to be limited to only that specific embodiment unless so indicated.

In addition, the steps and the ordering of the steps of methods described herein are not meant to be limiting. Methods comprising different steps, different number of steps, and/or different ordering of steps are also contemplated.

The above-described embodiments are intended to be examples only. Alterations, modifications and variations can be effected to the particular embodiments by those of skill in the art without departing from the scope, which is defined solely by the claims appended hereto.

What is claimed is:

1. An apparatus comprising:
at least one scanner transducer in communication with an anatomical scanner and configured to generate a plurality of anatomical scan images of a patient;
a tracking system comprising one or more sensors for tracking:
a position and orientation of the at least one scanner transducer; and
patient anatomical movement; and
a processor configured to receive signals from the tracking system and the plurality of anatomical scan images from the anatomical scanner, the processor further configured to apply image-processing based fusion to the plurality of anatomical scan images based on the tracked position and orientation of the at least one scanner transducer and the tracked patient anatomical movement to generate a fused scan image.

2. The apparatus of claim 1, further comprising:
an electrocardiogram (ECG) sensor configured to generate an ECG signal from the patient, and wherein the processor is further configured to time synchronize tracking information generated by the tracking system with the plurality of anatomical scan images based on the ECG signal.

3. The apparatus of claim 1 or 2, wherein the anatomical movement is respiratory movement.

4. The apparatus of claim 3, further comprising:
an electrocardiogram (ECG) sensor configured to generate an ECG signal from the patient,
wherein the processor is further configured to:
compute an overall average of the patient respiratory displacement based on the tracked patient respiratory movement during multiple previous R-R intervals in the ECG signal;
select a subset of the plurality anatomical scan images, each anatomical scan image in the subset having been taken during an R-R interval that has an interval average patient respiratory displacement that is within a predefined threshold of the computed average patient respiratory displacement;
generate the fused scan image from the selected subset of the plurality anatomical scan images.

5. The apparatus of claim 4, wherein the processor is further configured to:
compute an interval variance of the patient respiratory displacement based on the tracked patient respiratory movement during each R-R interval in the ECG signal, the variance being a difference between the maximum and minimum tracked displacement values within the given R-R interval, and
where each anatomical scan image in the subset having been taken during an R-R interval that has a computed variance patient respiratory displacement under a predefined variance value.

6. The apparatus of any one of claims 1 to 5, comprising:
an electrocardiogram (ECG) sensor configured to generate an ECG signal from the patient,
wherein the processor is further configured to:
select a subset of the plurality anatomical scan images, each anatomical scan image in the subset having been taken during a same subinterval of a respective R-R interval based on the ECG signal where the same subinterval corresponds to a particular phase of a heartbeat;
generate the fused scan image from the selected subset of the plurality anatomical scan images.

7. The apparatus of any one of claims 1 to 6, wherein the image-processing based fusion is a wavelet based image fusion.

8. The apparatus of any one of claims 1 to 6, wherein the image-processing based fusion is a random walker image fusion.

9. The apparatus of any one of claims 1 to 8, wherein the scanner transducer is an ultrasound transducer.

10. The apparatus of any one of claims 1 to 9, wherein the tracking system comprises at least one mechanical tracking system comprising at least one measuring arm for tracking at least one of the position of the scanner transducer and the anatomical movement of the patient.

11. The apparatus of claim 10, wherein the at least one measuring arm is configured for tracking the position and orientation of the scanner transducer, and the apparatus further comprises an optical tracking system comprising a plurality of cameras for tracking one or more patient markers positioned at the patient for tracking the patient anatomical movement.

12. The apparatus of any one of claims 1 to 9, wherein the tracking system comprises an optical tracking system comprising a plurality of cameras for tracking at least one of one or more scanner transducer markers positioned at the scanner transducer and one or more patient markers positioned at the patient for tracking the patient anatomical movement.

13. The apparatus of any one of claims 1 to 9, wherein the tracking system comprises an electromagnetic tracking system comprising a one or more electromagnetic sensors for tracking at least one of the position and orientation of the scanner transducer and the patient anatomical movement.

14. The apparatus of any one of claims 1 to 12, wherein alignment of the plurality of anatomical scan images during the generating of the fused scan image is performed independent of image data of the plurality of anatomical scan images.

15. The apparatus of any one of claims 1 to 14, configured to generate the fused scan image in the form of a three dimensional echocardiography image.

16. A method comprising:
generating a plurality of anatomical scan images of a patient with at least one scanner transducer;
tracking a position and orientation of the at least one scanner transducer during the generating;
tracking patient anatomical movement during the generating; and
applying image-processing based fusion to the plurality of anatomical scan images based on the tracked position and orientation of the at least one scanner transducer and the tracked patient anatomical movement to generate a fused scan image.

17. The method of claim 16, further comprising:
generating an electrocardiogram (ECG) signal from the patient; and
time synchronizing tracking information generated by the tracking system with the plurality of anatomical scan images based on the ECG signal.

18. The method of claim 16 or 17, wherein the anatomical movement is respiratory movement.

19. The method of claim 18, further comprising:
generating an electrocardiogram (ECG) signal from the patient;
computing an overall average of the patient respiratory displacement based on the tracked patient respiratory movement during multiple previous R-R intervals in the ECG signal;
selecting a subset of the plurality anatomical scan images, each anatomical scan image in the subset having been taken during an R-R interval that has an interval average patient respiratory displacement that is within a predefined threshold of the computed average patient respiratory displacement;
generating the fused scan image from the selected subset of the plurality anatomical scan images.

20. The method of claim 19, further comprising:
computing an interval variance of the patient respiratory displacement based on the tracked patient respiratory movement during each R-R interval in the ECG signal, the variance being a difference between the maximum and minimum tracked displacement values within the given R-R interval, and
where each anatomical scan image in the subset having been taken during an R-R interval that has a computed variance patient respiratory displacement under a predefined variance value.

21. The method of any one of claims 16 to 20, further comprising:
generating an electrocardiogram (ECG) signal from the patient;
selecting a subset of the plurality anatomical scan images, each anatomical scan image in the subset having been taken during a same subinterval of a respective R-R interval based on the ECG signal where the same subinterval corresponds to a particular phase of a heartbeat;
generating the fused scan image from the selected subset of the plurality anatomical scan images.

22. The method of any one of claims 16 to 21, wherein the image-processing based fusion is a wavelet based image fusion.

23. The method of any one of claims 16 to 21, wherein the image-processing based fusion is a random walker image fusion.

24. The method of any one of claims 16 to 23, wherein the plurality of anatomical scan images is generated with an ultrasound transducer.

25. The method of any one of claims 16 to 24, wherein the tracking of at least one of the position of the scanner transducer and the anatomical movement of the patient is performed using a measuring arm.

26. The method of claim 25, wherein the tracking of the position and orientation of the scanner transducer is performed using the measuring arm, the method further comprising tracking the anatomical movement of the patient using an optical tracking system comprising a plurality of cameras for tracking one or more patient markers positioned at the patient.

27. The method of any one of claims 16 to 24, wherein the tracking of at least one of the position and orientation of the scanner transducer and the anatomical movement of the patient is performed using an optical tracking system comprising a plurality of cameras for tracking at least one of one or more scanner transducer markers positioned at the scanner transducer and one or more patient markers positioned at the patient for tracking the patient anatomical movement.

28. The method of any one of claims 16 to 24, wherein the tracking of at least one of the position and orientation of the scanner transducer and the anatomical movement of the patient is performed using an electromagnetic tracking system comprising one or more electromagnetic sensors for tracking at least one of the position and orientation of the scanner transducer and the patient anatomical movement.

29. The method of any one of claims 16 to 27, wherein alignment of the plurality of anatomical scan images during the generating of the fused scan image is performed independent of image data of the plurality of anatomical scan images.

30. The method of any one of claims 16 to 27, configured to generate the fused scan image in the form of a three dimensional echocardiography image.

31. An apparatus comprising:
at least one scanner transducer in communication with an anatomical scanner and configured to generate a plurality of echocardiography scan images of a patient;
a tracking system comprising one or more sensors for tracking:
a position and orientation of the at least one scanner transducer; and
patient respiratory movement;
an electrocardiogram (ECG) sensor configured to generate an ECG signal from the patient; and
a processor configured to:
receive the plurality of echocardiography scan images from the anatomical scanner and signals from the tracking system;
time synchronize tracking information generated by the tracking system with the plurality of echocardiography scan images based on the ECG signal;
apply wavelet based image fusion to the synchronized plurality of echocardiography scan images based on the tracked position and orientation of the at least one scanner transducer and the tracked patient respiratory movement to generate a fused three dimensional echocardiography scan image.

32. A method comprising:
generating a plurality of echocardiography scan images of a patient with at least one scanner transducer;
tracking a position and orientation of the at least one scanner during the generating;
tracking patient respiratory movement during the generating;
generating an electrocardiogram (ECG) signal from the patient;
time synchronizing tracking information with the plurality of echocardiography scan images based on the ECG signal, the tracking information being generated by the tracking the position and orientation of the at least one scanner transducer and the tracking the patient respiratory movement; and
applying wavelet based image fusion to the synchronized plurality of echocardiography scan images based on the tracked position and orientation of the at least one scanner transducer and the tracked patient respiratory movement to generate a fused three dimensional echocardiography scan image.
